(19) United States
(12) Patent Application Publication
(10) Pub. No.: US 2004/0215161 A1
(43) Pub. Date: Oct. 28, 2004

(54) Disposable diaper that is easy to put on a wearer in a standing position

(76) Inventors: Yasuyuki Okuda, Haga-gun (JP); Haruko Toyoshima, Haga-gun (JP)

Correspondence Address: BIRCH STEWART KOLASCH & BIRCH, PO BOX 747, FALLS CHURCH, VA, 22040-0747, US

(21) Appl. No.: 10/626,547

(22) Filed: Jul. 25, 2003

(30) Foreign Application Priority Data
Jul. 26, 2002 (JP) 2002-218960
Jul. 26, 2002 (JP) 2002-218963
Jul. 26, 2002 (JP) 2002-218965
Feb. 4, 2003 (JP) 2003-027632
Feb. 4, 2003 (JP) 2003-027633

(51) Int. Cl. A61F 13/15; A61F 13/20

(52) U.S. Cl. 604/385.28

(57) ABSTRACT

A disposable diaper easy to put on a wearer in a standing position comprising a liquid permeable topsheet 2, a liquid impermeable backsheet 3, an absorbent member 4 interposed between the two sheets and having a pair of laterally extensible side parts on the below-waist portion of one longitudinal end section and a pair of standing gathers, wherein the region having the absorbent member in the crotch section contains a region having a lateral flexural stiffness of 25 gf/50 mm or less, the standing gathers are formed by fixing an elastic member at a fixing extension ratio of 100% or higher, and the standing gathers on each side, when extended in their non-fixed state, have a tensile load of from 20 to 120 gf at an effective extension ratio (fixing extension ratio—30%), and provides a tensile load increase rate of 1.0 gf/% or lower required to extend from a 20% extension ratio to the effective extension ratio.

### BACKGROUND OF THE INVENTION

The present invention relates to a fitted disposable diaper that is easy to put on a wearer, whether in a lying or standing position. It also relates to a fitted disposable diaper that is excellent in absorbency and is leak-proof.

Known disposable diapers include fitted type diapers having fastening tapes and pull-on diapers (also called pants type diapers or all-in-ones) that can be put on like ordinary underwear. Fitted disposable diapers are most commonly used because the diapers are applicable to children from newborn period, and also because of lower production cost.

Conventional fitted disposable diapers are generally designed to be put on a wearer such as a baby lying on its back. They are easy to put on a wearer who is lying still on its back. However, an active infant hardly keeps still in a lying position during diaper changing and often resists being made to remain still in a lying position. It is not easy to keep such an active infant in a lying position during diaper changing. When in particular an infant resists being diapered in a lying position and crawls about on all fours trying to escape being diapered, it is very difficult to conduct diaper changing. It is very likely that one may try to place a diaper on an infant while in a crawling or standing position, but such attempt may be met with great difficulty.

A fitted disposable diaper that can easily be placed on a wearer in a standing position while retaining excellent absorbing performance and leakproofness is not available yet.

Under these circumstances, one may think of using disposable pull-on diapers instead as their baby beings pulling up and/or stands but is very likely to be frustrated because it may still be practically impossible for one to make a baby, who has just started pulling up into a standing position to raise leg in order to put on a pull-on diaper. There may be no convenient way for one to do this but to resort to changing diapers while restraining an active baby or infant who is trying to escape from being diapered into usual lying position.

JP-A-6-63076 and JP-A-1-97201 disclose disposable diapers or underwear having an elastic band fixed to one of the longitudinal ends of the main body. In putting the diaper or underwear on a wearer, the elastic band is held around the wearer's waist, and an engageable part of the other longitudinal end is fastened to the elastic band. Since the elastic band of the proposed diaper or underwear is a closed loop, it is difficult to fit the band around the body of a standing baby by making the baby raise each leg. In short, a conventional disposable diaper cannot be seen as easy to put on a wearer whether the wearer is lying or standing.

### SUMMARY OF THE INVENTION

The present invention provides, in its first aspect, a disposable diaper which includes a liquid permeable topsheet, a liquid impermeable backsheet, a liquid retentive absorbent member interposed between the topsheet and the backsheet, and the diaper having a pair of longitudinal side portions and a pair of longitudinal end sections,

a fastening tape provided on each side edge of one of the longitudinal end sections thereof and a laterally extensible side part provided on each side area of a below-waist portion of the longitudinal end section having the fastening tapes, and standing gathers provided in each of the longitudinal side portions thereof, wherein

a region R having the absorbent member in the crotch section having a low stiffness region R1 having a flexural stiffness of 25 gf/50 mm or less in the diaper width direction of the diaper,

the standing gathers being formed by fixing an elastic member having a fixing extension ratio of 100% or higher, and

the standing gathers on each longitudinal side portion having tensile characteristics, which as measured in a state of the gathers not being fixed to the diaper, wherein the tensile load required to extend the standing gathers to an effective extension ratio, which is 30% lower than a fixing extension ratio is 2 from 0 to 120 gf, and the rate of increase of the tensile load required to extend from an extension ratio of 20% to the effective extension ratio is 1.0 gf/% or lower.

The present invention provides, in its second aspect, a disposable diaper for babies and infants that is easy to put on a wearer in a standing position, which includes a liquid permeable topsheet, a liquid impermeable backsheet, a liquid retentive absorbent member interposed between the topsheet and the backsheet, and a fastening tape provided on each side edge of one of a pair of longitudinal end sections thereof and having a pair of standing gathers and a pair of leg gathers each formed by fixing respective elastic members in each of a pair of longitudinal side portions thereof, wherein

a smallest width of the crotch section is from 100 to 240 mm,

a ratio of a distance W11 between a pair of fixed ends of the pair of standing gathers measured at the smallest width of a crotch section to a distance W12 between each elastic member that is arranged most outward in each of the respective leg gathers, and with both distances being measured at the smallest width of the crotch section, W11/W12, is from 0.67 to 0.81,

a distance W13 between each of the fixed ends of the pair of standing gathers and each of the elastic members that are arranged most outward in the pair of leg gathers as measured at the smallest width of the crotch section is smaller than a width W14 of the standing gathers on each side as measured at the smallest width of the crotch section,

the standing gathers being formed by fixing an elastic member having a fixing extension ratio of 100% or higher, and

the standing gathers on each longitudinal side portion having tensile characteristics, which as measured in a state of the gathers not being fixed to the diaper, wherein the tensile load required to extend the standing gathers to an effective extension ratio, which is 30% lower than the fixing extension ratio is from 20 to 120 gf, and the rate of increase of the tensile load required to extend from an extension ratio of 20% to the effective extension ratio is 1.0 gf/% or lower.

The present invention provides, in its third aspect, a disposable diaper that is easy to put on a wearer in a standing position, which includes a liquid permeable topsheet, a liquid impermeable backsheet, a liquid retentive absorbent member interposed between the topsheet and the backsheet, and a fastening tape provided on each side edge of one of a pair of the longitudinal end sections thereof, and a landing zone where the fastening tape can be fixed on an outer side of the other longitudinal end section of the pair, and a laterally extensible side part provided on each side area of a below-waist portion of the longitudinal end section having the fastening tapes, wherein

a tensile load required to extend the below-waist portion of the longitudinal end section having the fastening tapes in the diaper width direction to an extension ratio that is 30% lower than the maximum extension ratio is from 80 to 500 gf,

a smallest width of a crotch section of the diaper is from 100 to 240 mm, and

a region having the absorbent member in the crotch section has a bending stiffness of from 3 to 25 gf/50 mm in a width direction of the diaper.

The present invention also relates to a method of diapering a wearer that is in a standing position.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings in which like designations are used to designate substantially identical elements, and in which:

FIG. 1 is a perspective of a disposable diaper according to an embodiment of the first aspect of the present invention;

FIG. 2 is a plan of the disposable diaper of FIG. 1 with a part cut away, in which every elastic member is stretched substantially flat;

FIG. 3 is a plan showing the stiffness design of the diaper of FIG. 1;

FIG. 4 is a side view of the disposable diaper of FIG. 1 put on a standing infant with the first section B placed in the front;

FIGS. 5(a) through 5(d) show other stiffness designs applicable to a disposable diaper according to a first aspect of the present invention;

FIG. 6 is a graph showing the relationship between the extension ratio and the tensile stress (load) of the standing gathers of a disposable diaper embodiment in its non-fixed state;

FIG. 7 is a plan of a disposable diaper according to an embodiment of a second aspect of the present invention, in which every elastic member is stretched substantially flat;

FIG. 8 is a schematic cross-section of the disposable diaper of FIG. 7, taken along a line which the crotch section is narrowest and which corresponds to a centerline CL dividing the length of the diaper into two equal halves;

FIGS. 9(a) and 9(b) are each a plan of the first section B of a disposable diaper according to an embodiment of the present invention;

FIG. 10 is a plan of the disposable diaper according to an embodiment of a third aspect of the present invention, in which every elastic member is stretched substantially flat;

FIG. 11 is a schematic cross-section of the disposable diaper of FIG. 10, taken along a line which the crotch section is narrowest and which corresponds to a centerline CL dividing the length of the diaper into two equal halves; and

FIGS. 12(a) and (b) show examples of ways of placing the disposable diaper of FIG. 10 on a wearer who is in a standing position. The diaper is fastened on the wearer's stomach side in FIG. 12(a) and on the wearer's back side in FIG. 12(b).

### DETAILED DESCRIPTION OF THE INVENTION

All publication cited herein are hereby incorporated by reference.

The present invention related to a fitted disposable diaper that is easy to put on a wearer, whether the wearer is in a lying or standing position.

The present invention also relates to a fitted disposable diaper that is easy to put on a wearer, whether in a lying or standing position, and that is excellent in absorbency and is leak-proof.

Although not wanting to be limited by theory, the present inventors have several theories as to why diapering a wearer in a standing position is difficult. One reason is that the shape of the wearer's body on the stomach side changes between a lying position and a standing position. That is, the waist is larger in a standing position than in a lying position. For example, such waist size difference of an infant can be 5 cm. To facilitate diapering on an infant in a standing position, it is conceivable to increase the distance between a pair of fastening tapes on the diaper, namely the diaper width, thereby to cope with the change in waist size. In such case, however, the fastening tapes may overlap with each other when originally fixed on a wearer in a lying position.

Another reason is that one must hold the diaper onto the wearer's body while in the standing portion so as to not drop the diaper while fastening the tapes. In putting a diaper on a wearer in a lying position, the diaper is placed under the weight of the wearer's back so that the diaper hardly moves out of position under such weight.

Still another reason lies in that a conventional disposable diaper is designed to have a relatively large width in its crotch portion. Whereas the crotch width of a diaper is not a large consideration regarding the ease of diapering a wearer in a lying position with its legs spread apart, a diaper with a wide crotch portion is difficult to put on a wearer in a standing position due to the difficulty in placing a diaper through a narrow space between the wearer's legs. A disposable diaper with a reduced width in the crotch portion could be placed on a wearer in a standing position with more ease, but it has been difficult to design a reduced crotch portion width without sacrificing the absorbency and leak-proof properties that are essential requirements of diapers.

Where, in particular, a fitted disposable diaper having a pair of standing gathers (also called cuffs) and a pair of leg gathers on each longer side portions thereof is designed to have a reduced width in its crotch portion, either the distance between the pair of standing gathers or the distance between each standing gathers and the leg gathers on each side may be reduced out of necessity. Reduction of the former distance often results in insufficient absorbing performance. Reduction of the latter distance often results in the insufficient formation of a pocket between the standing gathers and the leg gathers, which can cause discharged body waste to flood over and leak.

In putting a conventional fitted disposable diaper on a wearer in a lying position, the portion having a pair of fastening tapes goes on the wearer's back-side and thus the diaper is held between the body and the bed or changing area. Therefore the other portion of the diaper where a landing zone is provided can easily be held to the wearer's stomach, and it is easy to adhere the fastening tapes, which naturally come near the landing zone, onto the landing zone. In contrast, when the diaper is put on a wearer in a standing position, one must hold the portion having the fastening tapes to the wearer's back while simultaneously fastening the fastening tapes on the landing zone of the diaper on the wearer's front side. It is very difficult for one to do this alone without help.

The present invention will be described in detail based on its preferred embodiments.

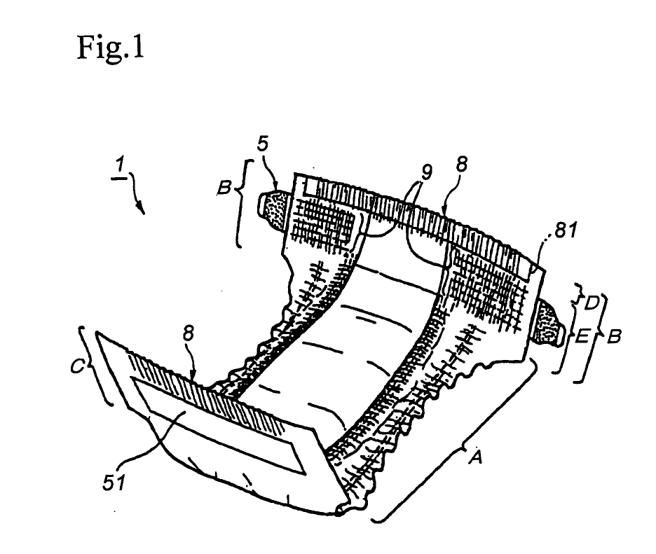

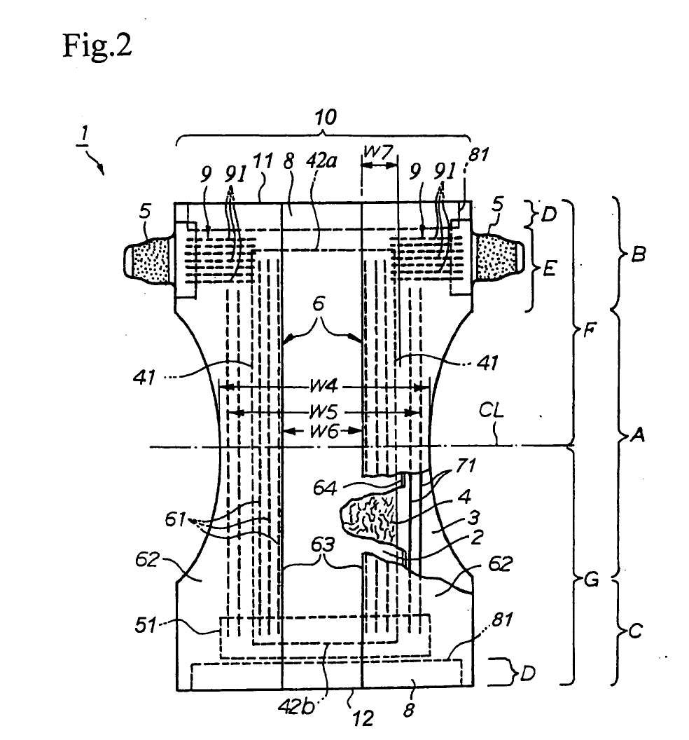

A preferred embodiment of the disposable diaper according to the first aspect of the present invention (hereinafter referred to as a first embodiment) is shown in FIGS. 1 to 3. The diaper 1 shown in FIGS. 1 and 2 comprises a liquid permeable topsheet 2, a liquid impermeable backsheet 3, and a liquid retentive absorbent member 4 interposed between the topsheet 2 and the backsheet 3. The disposable diaper 1 is of a fitted type. It is divided crosswise into a crotch section A which is in the middle lengthwise, a first section B on one side of crotch section A (one of the longitudinal end sections), and a second section C on the other side of crotch section A (the other longitudinal end section). The first section B has a pair of fastening tapes 5 provided on each side edge thereof, and the second section C has on its outer surface a landing zone 51 for receiving the fastening tapes 5. The crotch section A is placed along the crotch of a wearer. Either the first section B or second section C may be placed on the wearer's back-side, and the other along the wearer's front (the wearer's stomach side).

The crotch section A has an inward arc on both sides thereof. The disposable diaper 1 as a whole has a sand or hour glass-shape with its longitudinal middle portion narrowed.

The topsheet 2 is substantially rectangular and larger than the absorbent member 4 when viewed from above. The absorbent member 4 is arranged in the middle of the width of backsheet 3. The backsheet 3 is sand or hour glass-shaped in agreement with the contour of diaper 1.

The topsheet 2 and the backsheet 3 both extend outward from the two longer sides 41 and two shorter sides 42a and 42b of the absorbent member 4 and are bonded together at these extensions. The backsheet 3 extends outward from the two longer sides of the topsheet 2.

A pair of standing gathers (also called cuffs) 6 is lengthwise provided on both longitudinal side portions of the diaper 1. The standing gathers 6 on each side are formed of a gather-forming sheet 62 having elastic members 61, the gather-forming sheet 62 being disposed to cover both side areas of the longer side edge of the topsheet 2. Each sheet 62 is fixed to the topsheet 2 in a straight line pattern parallel to the diaper longitudinal direction by known bonding means such as a heat seal or an adhesive. The straight line is positioned between the longer side edge 41 of the absorbent member 4 and the laterally most inward leg elastic member 7 (described later). The linear joint of the sheet 62 with the topsheet 2 is a fixed end 64 of the standing gathers 6. The part of the sheet 62 extending outward from the fixed end 64 is fixed to the topsheet 2 or the backsheet 3. In the areas near the two longitudinal ends of the diaper 1, the part of the sheet 62 extending inward from the fixed end 64 is fixed to the topsheet 2. The elastic members 61 used to form the standing gathers 6 are elastic threads, which are arranged per side substantially in parallel with the free end 63 of the standing gathers 6.

The first section B has a waist portion D and a below-waist portion E. An elastic member 81 is provided in the waist portion D to form an extensible waist part 8. A plurality of elastic members 91 are provided on each side area of the below-waist portion E to form a pair of laterally extensible side parts 9. Both of the extensible side parts 9 are arranged in the area between the pair of fastening tapes 5.

The second section C also has an extensible waist part 8 in its waist portion D. The two waist portions D constitute the longitudinal end parts of the diaper 1 and are applied to the wearer's waist. More specifically, an area from the longitudinal end 11 or 12 to 20 mm inward is called a waist portion D. The diaper 1 according to a first embodiment has the extensible waist part 8 formed by the waist elastic member 81 in the waist portion D in both the first and second sections B and C. The waist elastic member 81 in each waist portion D is fixed between the backsheet 3 and the topsheet 2 or the sheet 62 in its stretched state across the diaper. The waist elastic member 81 used in this embodiment is a strip of urethane foam.

FIG. 2 is a plan view of the disposable diaper 1 with all its elastic members in their stretched state; the upper part of the diaper 1 is designated first section B, and the lower part second section C. The below-waist portion E of the first section B is a portion located below the waist portion D (20 mm wide from the top end 11 downward) and above the crotch section A (the portion applied to the crotch of a wearer and describing an inward arc on both sides thereof in conformity with the wearer's thighs). The plurality of below-waist elastic members 91 are arranged per extensible side part 9 in parallel to the width of the diaper at prescribed intervals.

The below-waist elastic members 91 are fixed in their stretched state such that elastic extensibility may develop in at least areas extending outward from both side edges 41 of the absorbent member 4 in the direction of the width of the diaper. The below-waist elastic members 91 are not disposed in the middle portion of the area between the longer side edges 41 of the absorbent member 4. The elastic members 91 are fixedly sandwiched in between two soft sheets (not shown) which are provided in the first section B over the width of the main body 10 (see FIG. 2). The term “main body 10 of the diaper 1” as used herein means the part composed of the topsheet 2, the backsheet 3, and the absorbent member 4 and including all sections A to C. The two sheets are fixed by bonding between the backsheet 3 and the topsheet 2 or the gather-forming sheet 62. The inward end of each below-waist elastic member 91 is positioned slightly inward from the side edge 41 of the absorbent member 4. Therefore, there is no elastic member 91 placed in the area from the middle of the width of the absorbent member 4 up to a position near each side edge 41.

The diaper 1 also has a pair of leg gathers on each longitudinal side thereof. The leg gathers on each side are formed by arranging a plurality of leg elastic members 71 in parallel with the diaper length.

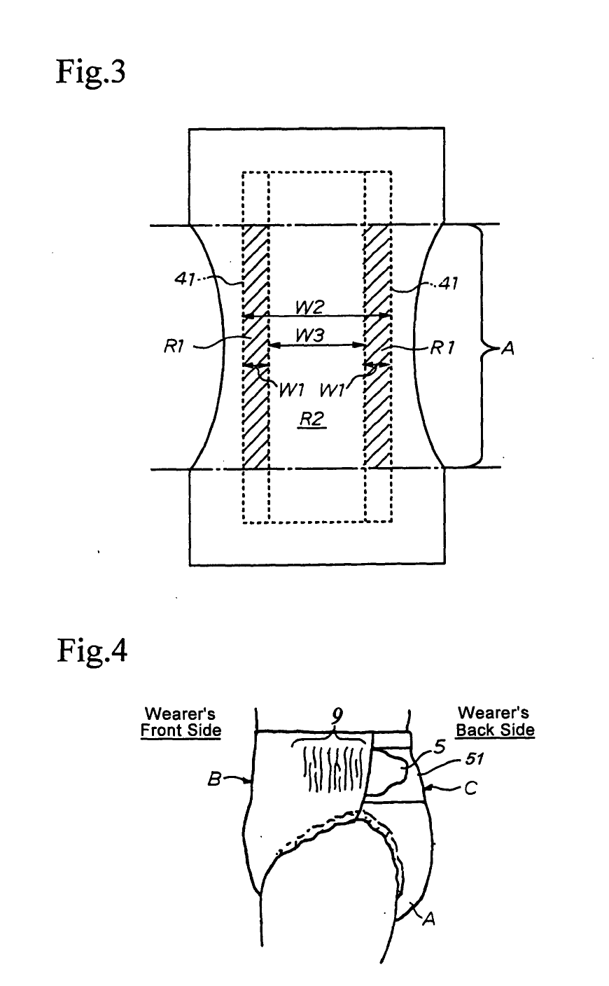

As shown in FIG. 3, the diaper 1 according to a first embodiment is characterized by regions R1 having a lateral flexural stiffness of 25 gf/50 mm or lower in the region having the absorbent member 4 in the crotch section A. More specifically, in the crotch section A, the region where the absorbent member 4 is disposed (hereinafter referred to as the region R) contains regions R1 indicated by slant lines in FIG. 3 which are less stiff (softer) than the other region R2 in the region R. The region R1 will hereinafter be called a low stiffness region R1. The low stiffness regions R1 have a lateral (in the diaper width direction) flexural stiffness of 25 gf/50 mm or lower. The term “region” as in “the region having the absorbent member 4 in the crotch section A (region R)” or as in “the low stiffness region” as used herein means a three-dimensional part containing the absorbent member 4 in its thickness (between the upper and lower surfaces of the diaper).

In a first embodiment, each of the low stiffness regions R1 is an oblong region provided along each longer side edge of the absorbent member 4.

The region R in the crotch section A is required to have sufficient width and space for collecting and retaining body waste within the crotch section while being worn and should have a minimum restoring force for maintaining such width and space. In diapering a baby in a standing position, when two ends of the diaper that are passed between legs are pulled up in the front and the back, it is desirable for the diaper itself to fit the crotch snugly and not to slide down, in order to facilitate fastening. Considering these factors, the lower limit of the flexural stiffness of the low stiffness region R1 is preferably about 3 gf/50 mm, still more preferably about 5 gf/50 mm. For assuring ease of diapering without sacrificing fit and safety against leakage, a preferred flexural stiffness of the low stiffness region R1 is 25 gf/50 mm or lower, more preferably from 3 to 25 gf/50 mm, and even more preferably from 5 to 20 gf/50 mm.

In the diaper 1 of a first embodiment, the region R also has a region R2 having a width W3 (see FIG. 3) of preferably at least 50 mm across the diaper and a lateral flexural stiffness of higher than 25 gf/50 mm (hereinafter referred to as a high stiffness region R2). From the standpoint of ease of diapering and shape retention during use, a preferred upper limit of the flexural tiffness of the high stiffness region R2 is 60 gf/50 mm, and a more preferred flexural stiffness of the high stiffness region R2 is from 30 to 50 gf/50 mm.

To improve ease of diapering a wearer in a standing position, the width W1 (see FIG. 3) of each low stiffness region R1 preferably ranges from 15 to 50% of the width W2 (see FIG. 3) of the region R, and the total width of the low stiffness regions R1 preferably ranges from 30 to 100% of the width W2. The width W3 (see FIG. 3) of the high stiffness region R2 between the pair of the low stiffness regions R1 is preferably from 0 to 70% of the width W2. The width W2 is preferably 70 mm or longer in order to secure sufficient absorbency of the crotch section. The region R width W2 of 70 mm or longer is also effective in facilitating the fastening of a diaper while a wearer is in a standing position because, when the diaper is inserted between the wearer's legs and pulled upwards, the crotch section fits the wearer's crotch well and does not readily slide down thereby making it easy for one to fasten tapes. From the standpoint of ease of diapering and proper fit to the wearer's crotch, the width W2 is preferably 150 mm or shorter. The width W3 is preferably from 0 to 80 mm. The smaller the width W3, the better for ease of diapering and obtaining a snug fit.

The lateral (in the width direction of the diaper) flexural stiffness of the low stiffness region R1 and the high stiffness region R2 is measured as follows.

Measurement of Flexural Stiffness:

Standing gathers are removed from a diaper. A 50 mm wide and 80 mm long rectangular specimen containing all the constituent members including from the topsheet to the backsheet in its thickness direction is cut out of the region R. The width (50 mm) and the length (80 mm) of the specimen are in the length and the width directions of the diaper, respectively. Care should be taken so that the specimen has substantially no crease or wrinkle that may influence the measurement.

Measurement is carried out with a Tensilon tester, RTC-1150A supplied by Orientec, equipped with a 5-kg load cell (range: 200 gf) in accordance with JIS K7171 (Plastics-Determination of flexural properties) (R1=5.0±−0.1 mm, R2=±0.2 mm). The specimen is placed on two supports 50 mm apart with its length being along the line connecting the two supports, and an indenter is positioned to just touch the mid-point of the specimen. The indenter is moved down at a speed of 30 mm/min to obtain a load-deformation curve. The maximum of the bending stress is taken as the flexul stiffness (gf/50 mm). The “50 mm” in the unit corresponds to the width of the specimen bent by the indenter.

Where the region R1 or R2 is greater than 50 mm across the diaper, a specimen is cut such that the whole width of the region is contained in the specimen length. Where the region R1 or R2 is equal to or shorter than 50 mm across the diaper, a specimen is cut out so as to contain the whole width of the region in the specimen length and is placed over the supports with at least part of that region positioned between the supports. For instance, where a low stiffness region R1 having a width of 50 mm or smaller is arranged on each side of the region R, a specimen of the region R1 is cut out such that the low stiffness region R1 is present in one longitudinal end portion of the specimen and placed on the supports such that one end of the low stiffness region is positioned at the edge of one support and the other end between the supports. In this case, the mid-point of the specimen, at which the indenter presses, may be outside of the low stiffness region R1. Nevertheless the measured maximum load is regarded as the flexural stiffness (gf/50 mm) of the low stiffness region because the maximum load is predominantly governed by the stiffness of the low stiffness region.

Where a specimen containing a region whose width exceeds 50 mm in the diaper width direction shows a maximum load exceeding 25 gf/50 mm as measured in the same manner as described above, the region R has a high stiffness region R2 having a width W3 (see FIG. 3) of 50 mm or larger and a lateral flexural stiffness exceeding 25 gf/50 mm.

The low stiffness region R1 in a first embodiment is formed by varying the constitution of part of the absorbent member 4 within the region R from the constitution of the other part, that is, by reducing the basis weight of part of the absorbent member 4 as compared to that of the other part.

Methods of reducing the basis weight of part of an absorbent member from that of the other part include the following; (1) An absorbent member is fabricated of an upper absorbent layer and a lower absorbent layer, part of which is made solely of either the upper or the lower absorbent layer to provide a low stiffness region; (2) An absorbent member is fabricated of fibers air-laid by suction on a net, part of which has a smaller air-laid fiber content than the other part; (3) An absorbent member is fabricated of three or more absorbent layers, part of which has a smaller number of absorbent layers by at least one layer from the other part; (4) An absorbent member is fabricated of two or more absorbent layers, part of which lacks one or more constituents. Preferred is method (1) because the upper and lower absorbent layers are separately prepared, which is advantageous in realizing the absorbing performance and the basis weight as designed

In addition to making a difference in the basis weight, the low stiffness region can also be formed by other methods, such as (a) slitting the absorbent member 4, (b) discretely arranging separate pieces of an absorbent member, (c) subjecting part of an absorbent member to embossing or like treatment, (d) disposing or incorporating fibers having compressive elasticity, (e) eliminating an adhesive (e.g., a hot-melt adhesive), and (f) eliminating one or more members making up the diaper.

The standing gathers 6 are formed by fixing a low modulus elastic member at a high extension ratio. The extension ratio at which an elastic member is fixed will hereinafter be referred to as a fixing extension ratio. In the present embodiment, the elastic member for standing gathers is fixed in a stretched state at a fixing extension ratio of from 100% or higher, preferably from 100 to 300%, more preferably from 130 to 200%.

If the fixing extension ratio of the elastic member is less than 100%, the resultant gathers do not rise up sufficiently in a fitted diaper. It follows that the standing gathers 6 may fall or readily bend when the free end of the standing gathers 6 is held to the wearer's crotch particularly in diapering a wearer in a standing position, making correct diapering difficult. Besides, when a wearer changes its position, a gap may be formed between the free end of the gathers and the skin, through which body waste can leak.

If the fixing extension ratio is more than 300%, the disposable diaper may curl up excessively and be difficult to put on. Such a curling diaper may be particularly difficult to put on a wearer in a standing position because the diaper put through between the legs will slide down easily before it can be fastened. If the diaper has already begun to slide down when one manages to fasten the diaper, this results in poor fit and impaired leak-proof properties. As long as the fixing extension ratio is 300% or less, the diaper is preferably prevented from curling up and is easy to put on. In diapering a wearer in a standing position, since the diaper hardly slides down between legs while fastening with the fastening tapes, the diaper can easily be put on in the right position without bunching to provide a good fit and have satisfactory leak-proof properties.

The fixing extension ratio of the standing gathers 6 is measured as follows.

Measurement of Fixing Extension Ratio:

A strip of the gathered forming sheet 62 having an elastic member is cut off along the diaper length direction so that the strip (specimen) may contain at least the lengthwise middle of the gathers. The specimen is stretched to the maximum, i.e., to the stretched state as shown in FIG. 2. A distance between arbitrarily chosen two points, preferably about 200 mm apart, of the stretched specimen is taken as a stretched length H. The specimen is then put into its natural state, i.e., let to contract spontaneously, and a distance between the same two points (natural length h) is measured. The fixing extension ratio is represented by equation (1):

Fixing Extension ratio (%)=[(H−h)/h]×100 (1)

The natural length h is measured by placing the gathered strip on a flat surface while minimizing the unevenness due to gathering with minimized load applied thereto.

The tensile characteristics of the standing gathers 6 (either one of the pair) are such that the tensile load required to extend the standing gathers 6 in its non-fixed state (the state not fixed to the diaper, that is, the state after being cut off the diaper) to an effective extension ratio (equivalent to the value which is 30% lower than the fixing extension ration) is from 20 to 120 gf, preferably from 50 to 100 gf, and that the increase rate of tensile load required for extending in the non-fixed state from an extension ratio of 20% up to the above-identified effective extension ratio is 1.0 gf/% or lower, preferably 0.7 gf/% or lower.

Although not wanting to be limited by theory, the reason why the tensile characteristics of the standing gathers are evaluated at the effective extension ratio, (e.g. the fixing extension ratio—30%), is that the extension (%) vs. load curve often contains the tensile load component attributed to the constituent members of the standing gathers in the region above the effective extension ratio. The part of the curve above the effective extension ratio is likely to represent a tensile stress in excess of the physical properties of the gathers per se, that is, false data. In addition, a fitted diaper with standing gathers is curved into a U-shape when put on, and the free end of the standing gathers disposed on the inner side of the diaper depicts a U-shape having a shorter length than that of the outer surface of the U-shaped diaper. Since the standing gathers are rarely stretched to the fixing extension ratio in actual use, it can be seen as reasonable to discuss the behavior of the standing gathers at a given extension ratio lower than the fixing extension ratio in evaluating the behavior during an actual use.

The tensile characteristics (tensile load and tensile load increase rate) of the standing gathers at the effective extension ratio in a non-fixed state are measured as follows.

Measurement of Tensile Characteristics:

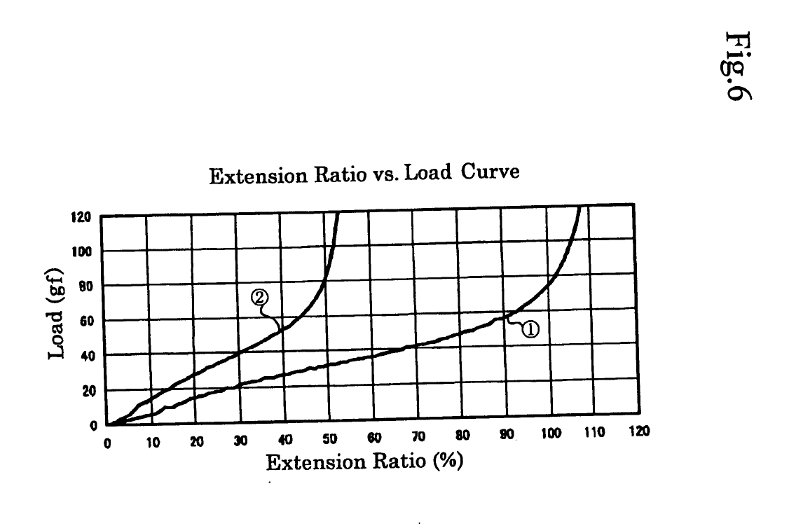

A strip of the gathered sheet 62 having an elastic member is cut off at its fixed end 64 from the part containing at least the lengthwise middle of the standing gathers 6 to prepare a specimen. The specimen should have a natural length of at least 70 mm as measured on a flat surface while minimizing the unevenness due to gathering with minimized load applied thereto. It is desirable to use the same specimen used in the measurement of the fixing extension ratio. The specimen is clamped in the jaws of a Tensilon tensile tester, RTC-1150A supplied by Orientec, equipped with a 5 kg-load cell. The jaws are set at an initial distance of 50 mm, which is an initial natural length of the specimen at 0% extension. The specimen is extended up to the fixing extension ratio at a rate of 300 mm/min, and the extension ratios vs. increasing tensile load are plotted to obtain a curve with the extension ratio (%) as abscissa and the tensile load (gf) ordinate (see FIG. 6).

The tensile load at an extension ratio of 20% and an effective extension ratio are read from the curve. The slope of the curve of from the extension ratio of 20% to the effective extension ratio, represented by (tensile load at effective extension ratio-tensile load at 20% extension ratio)/(effective extension ratio (%)—20%), is calculated to obtain a tensile load increase rate (gf/%) of from 20% extension ratio to effective extension ratio.

If the tensile load at the effective extension ratio is less than 20 gf, the gathers do not rise up sufficiently in a fitted diaper. It follows that the standing gathers may fall or readily bend when the free end of the standing gathers is held to the wearer's crotch particularly in diapering a wearer in a standing position, making correct diapering difficult. Besides, when a wearer changes position, a gap may be formed between the free end of the gathers and the skin, through which body waste can leak. Even when the free end of the pair of standing gathers is in close contact with the wearer's body, the squeezing force of the standing gathers is so weak that the force of shutting discharged body waste in the space between the pair of standing gathers would be insufficient. It is likely to follow that the body waste edges out over the free end of the standing gathers. Therefore, the standing gathers with such a low tensile load cannot be seen as effective against leakage.

If the tensile load at the effective extension ratio is more than 120 gf, the U-shaped diaper needs a large force to open the diaper wide and may be difficult when placing a diaper on a wearer lying on its back. When changing diapers after being soiled, the diaper is difficult to handle due to its strong tendency to curl up. Furthermore, in diapering a wearer in a standing position, the diaper placed between the legs may roll up or slide down, making fastening difficult.

These problems occur when the elastic member is fixed at a high extension ratio as with the case of embodiments of the present invention. In other words, the inconvenience arises from the phenomenon that the standing gathers stretched in diapering exhibit contractibility to be relieved from the tensile stress and make the whole diaper roll-up. Solving such problems, the present invention has succeeded in assuring ease of diapering by reducing the tensile load to some extent thereby suppressing its behavior to contract, retarding the contraction, or reducing the force necessary to widely open the contracted gathers. With the fixing extension ratio being equal, the tensile stress is a governing factor. The smaller the tensile stress, the more extensible the standing gathers and the easier to handle the diaper. Unless the tensile load at the effective extension ratio exceeds 120 gf, the manageability of the diaper is not impaired, and a one is able to widely open the diaper with ease.

The tensile load increase rate is desirably as small as possible. With a smaller increase rate, the force of the standing gathers to come into intimate contact with the skin and to block body waste will be less variable depending on the wearer's position or the way of diapering. Further, the diaper will be easier to handle, giving no feeling of abrupt change in force necessary to widely open the diaper (same as widely open). As a result, the diaper will be easier to put on whether a wearer is in a lying position or a standing position.

Accordingly, it is desirable that the tensile load increase rate, calculated as an increase in tensile load per unit increase in extension ratio, be 1.0 gf/% or lower at every point between 0% extension ratio up to an effective extension ratio. The tensile load increase rate may exceed 1.0 gf/% in some regions between 0% extension ratio up to an effective extension ratio. A diaper showing such a tensile load increase rate as exceeding 1.0 gf/% is not deemed to be a departure from the scope of the present invention as long as such a high increase rate is observed in the region of from 0% extension ratio to 20% extension ratio. That said, the upper limit of the tensile load increase rate in the region from 0 to 20% extension is preferably 2.0 gf/%.

This is the reason the tensile load increase rate in the region from 20% extension to an effective extension ratio is specified in the present invention.

The disposable diaper 1 according to a first embodiment has a low stiffness region R1 as part of the region R (the region having the absorbent member 4 in the crotch section A) and a pair of low-modulus standing gathers 6 provided at a high extension ratio. This design renders the diaper 1 extremely easy to put on a wearer in a standing position as well as a lying position.

A conventional fitted type disposable diaper is relatively easy to put on a wearer (e.g., a baby) lying on its back because one can easily spread the wearer's leg wide. In diapering a wearer in a standing position, on the other hand, since it is difficult to spread the wearer's leg wide while standing up, one must place a disposable diaper, which is made up of so many members, through a narrow space between the legs. Probably because of this, the position of a disposable diaper that has been put on a standing wearer is mostly below the intended position. In other words, the front waist size of the diaper that has been put on a wearer when the wearer is standing is larger than the true front waist size of the wearer. One has difficulty in diapering a wearer in a standing position with a conventional fitted type disposable diaper as compared with diapering a wearer in a lying position because of not only the change in the front waist size of a wearer depending on whether the wearer is standing or lying but also the change of the front waist size of the diaper with the change of the position where the waist portion of the diaper is held against.

To solve this problem, it might be considered effective to reduce the lateral stiffness of a disposable diaper. However, a diaper so designed undesirably bunches up in its crotch portion and may leak. Therefore, such an approach seems to be impractical.

According to a first embodiment, the standing gathers preferably have a high extension ratio and a low modulus and therefore rise sharply and firmly. Even if the crotch portion bunches, the standing gathers secure space enough to substantially prevent leakage. The diaper of this embodiment can be smoothly placed through the narrow space between the legs of a wearer in a standing position and held to the body at the same correct position as in diapering a wearer in a lying position. Thus, the present embodiment is both leak-proof and easier to diaper a wearer in a standing position. With the ease of diapering a standing wearer being so improved, it is no longer necessary to largely increase the extension length of the first section B or the width of the first section B, so that there is no impairment of ease in diapering a wearer in a lying position.

The standing gathers of the present invention can be formed by, for example, adhering at least one elastic member 61 to a gather-forming sheet 62, such as a nonwoven fabric. The elastic member or members have a tensile load increase rate of 1.0 gf/% or less from 20% extension ratio to an effective extension ratio and a tensile load of from 20 to 120 gf at the effective extension ratio as a whole. The term “as a whole” as used herein means “as a total of two or more elastic members”. The elastic members for standing gathers include elastic threads (e.g., rubber threads, preferably those having a thickness of 450 dtex or smaller), elastic strips (e.g., rubber strips, preferably those having a thickness to width ratio of 0.1 to 1), and elastic films. Materials of the elastic members include natural rubber, synthetic rubbers (e.g., styrene-butadiene, butadiene, isoprene or neoprene rubbers), ethylene-vinyl acetate copolymers, extensible polyolefins, and urethane rubber. In using a plurality of elastic members, it is preferred for each of them to have a tensile load increase rate of 0.5 gf/% or lower from 20% extension ratio to the respective effective extension ratio and a tensile load of 5 to 50 gf at the effective extension ratio.

The disposable diaper 1 of the first embodiment has the below-waist elastic members 91 fixedly held between two sheets integrated with the main body 10 of the diaper 1. The main body 10 is thus provided with elastic extensibility as desired. Unlike the configurations in which a separately prepared elastically extensible member is joined to each side of the main body of a disposable diaper as disclosed in JP-T-9-507409 and JP-A-6-63077, the extensible side parts are prevented from being broken even when the fastening tapes 5 are pulled strongly in cases, for example, where one hastily places a diaper on an infant crawling about and trying to escape being diapered. The diaper 1 is therefore easy to put on a wearer either from the front or the back of the wearer. That is, one can put the diaper 1 under the back of a wearer lying on its back (facing the person placing the diaper) and adhering the fastening tapes 5 to the landing zone 51 on the stomach side (this way of diapering will be sometimes called “a back-to-front way of diapering”), or one can put on the diaper 1 backward and adhering the fastening tapes on the back side of the wearer (this way of diapering will be sometimes called “a front-to-back way of diapering”).

In the disposable diaper 1 of the first embodiment, two portions on opposite sides of the centerline CL (see FIG. 2) dividing the length of the diaper 1 in equal halves, designated as sections F and G, preferably have a saturated absorption capacity ratio of from 45/55 to 55/45. By so designing, the diaper 1 does not substantially leak even when put on in a front-to-back way of diapering. The diaper 1 is not only suited to a back-to-front way of diapering but also capable of being put on in a front-to-back way with ease and does not leak whichever way of diapering is chosen. In order to substantially prevent leakage irrespective of the way of diapering, the F/G ratio of saturated absorption capacity preferably ranges from 48/52 to 52/48, and the difference in saturated absorption capacity between the F and G sections is preferably as small as possible. In FIG. 4 is shown the disposable diaper 1 put on an infant in a standing position with the first section B on the stomach (in a front-to-back way).

The saturated absorption capacity of the section on each side of the centerline CL is measured as follows.

Method of Measuring Saturated Absorption Capacity:

A diaper 1 is cut across into two along the centerline CL, the line dividing the length of the diaper into equal halves. The standing gathers, the leg gathers, and the extensible side parts in the below-waist portion are cut off from each half. Care should be taken to keep the structure of the absorbent member intact. The cut piece is placed on a metal net which is configured to be taken out of a container horizontally. The metal net with the cut piece on is weighed and then put in a container having a drain hole in the lower part of one side thereof. The drain hole is closed, and 0.9 wt % physiological saline is poured to completely immerse the absorbent member at a rate controlled so that polymer particles and the like may not fall off from the cut edge of the diaper. After completely immersed, the cut piece is allowed to stand for 30 minutes. The container is tilted 10° with the drain hole side down, and the drain hole is opened to discharge the liquid. The container is kept tilted for 30 minutes until the liquid is completely drained. The metal net with the cut piece on is taken out of the container and weighed again. The difference between the weight after immersion and the weight before immersion is taken as the saturated absorption capacity (g) of the cut piece.

Each of the extensible side parts 9 preferably has a natural length of 20 mm or more, more preferably from 20 to 100 mm, as measured in the diaper width direction in the state removed from the diaper and contracted. The tensile characteristics of the pair of extensible side parts 9 are preferably such that, when the fastening tapes 5 are pulled apart in the diaper width direction to extend the extensible side parts 9, the tensile load at a middle extension length is 30 to 300 gf. The tensile load at a middle extension length is measured as follows. The fastening tapes are clamped in the jaws with the first section B being in its natural state (i.e., contracted state) and pulled to extend the first section B to a length of 95% of the maximum extension length. The tensile load required to extend to the mid-point between the natural length and the maximum extension length is read.

The diaper 1 is easy to put on since the natural length of each extensible side part 9 in the diaper width direction is 20 mm or greater. In particular, it is easy to fix the fastening tapes to the landing zone in putting the diaper 1 on a wearer in a standing position and a wearer not facing the person trying to place the diaper. The diaper 1 is capable of coping with the change in front waist contour and size to impose a moderate squeezing force.

Where the tensile stress at the middle extension length of the first section B is 30 gf or greater, the diaper 1 offers a good fit to the wearer's below-waist portion. The diaper is prevented from sliding down, losing its shape, or making a gap through which body waste may leak and clothes may enter. Where the tensile stress at the middle extension length is 300 gf or smaller, the squeezing force is controlled moderately such that a wearer does not feel tightness. The extensible side parts can be extended by a moderate force, which makes diapering easy. This is particularly advantageous for diapering a wearer in a standing position or adhering the fastening tapes to the landing zone on the back of a wearer who is crawling.

To ensure the improvement of ease to put the diaper on a wearer in a standing or lying position, especially a wearer in a standing position, it is preferable that the smallest width W4 (see FIG. 2) of the diaper 1 in the crotch section be from 100 to 240 mm, preferably from 120 to 230 mm, still even more preferably from 120 to 220 mm, and that the distance W5 (see FIG. 2) between the leg elastic member 71 that is the most outward on one side of the diaper and that on the other side preferably be from 90 to 230 m, more preferably from 100 to 220 mm, still even more preferably from 100 to 200 mm. To assure ease of putting the diaper between the wearer's legs and pulling the diaper upward, it is preferred that the distance W6 (see FIG. 2) between the free ends of the paired standing gathers 6 is 100 mm or smaller, particularly 80 mm or smaller. The width W7 (see FIG. 2) of the standing gathers on each side is preferably 25 to 60 mm.

With considerations given only to diapering a wearer in a lying position, the narrowing of the width of the crotch section, e.g. the target zone where urine is going to be provided, leads to leakage. In the first place, a wide crotch section is no hindrance to diapering a lying wearer. Diaper manufacturers would not generally think of narrowing the crotch width. On the other hand, the present inventors have revealed the need of disposable diapers that is easy to put on not only a lying baby but a standing baby without raising the baby's leg one by one. In other words, the inventors have studied disposable diapers suited to those wearers who have mastered crawling and are able to pull themselves up on furniture and walk around holding on to it and are now moving on to become toddlers. To meet this need, the inventors have reached an idea that the crotch width could be reduced for improving ease of diapering. This idea has been realized by providing low modulus standing gathers for securing high safety against leakage while being worn. The inventors have thus succeeded in improving the ease of diapering a wearer in a standing position without sacrificing leakage, whether the diaper has been put on a wearer in a standing position or lying position.

Materials of the members making up the disposable diaper 1 according to a first embodiment will be described below.

The materials for forming the topsheet 2, the backsheet 3, the standing gathers-forming elastic members 61, the standing gather-forming sheet 62, and the landing tape for forming the landing zone 51, and the like are not limited and selected appropriately from known materials commonly used in conventional disposable diapers.

The absorbent member 4 includes one made of a fiber aggregate and one comprising a fiber aggregate and a superabsorbent polymer. The fiber aggregate includes nonwoven fabrics or fiber webs prepared by various processes. In using a superabsorbent polymer, the polymer may be dispersed in the interstices of a fiber aggregate or sandwiched in the form of a layer between nonwoven fabrics or fiber webs made of fibrous materials. The absorbent member comprising a fiber aggregate or a combination of a fiber aggregate and a superabsorbent polymer is preferably wrapped in a soft sheet of paper or liquid-permeable nonwoven fabric.

The fastening tape 5 includes a tape having a male member (hook) of a mechanical fastener and a tape having a self-adhesive layer formed by applying a self-adhesive. It is likely that a fastening tape with a self-adhesive layer sticks to a one's hand, making it difficult to carry out fastening in a front-to-back way. To avoid this, fastening tapes that can be mechanically engaged on the landing zone 51, such as those having a male member of a mechanical fastener, are preferred especially for a front-to-back way of diapering. Where the outer surface of the backsheet 3 is made of an engageable material such as a nonwoven fabric, that surface can serve as a landing zone 51 for the mechanical fasteners.

The waist elastic members 81, the below-waist elastic members 91, and the leg elastic members 71 are known and conventional. Useful forms of these elastic members include threads (e.g., rubber threads), tapes or strips of a certain width (e.g., rubber strips), and films. Materials of the elastic members include natural rubber, synthetic rubbers (e.g., styrene-butadiene, butadiene, isoprene or neoprene rubbers), ethylene-vinyl acetate copolymers, extensible polyolefins, and urethane rubber.

The waist elastic member 81 is preferably an elastic strip having a predetermined width. The below-waist elastic members 91 are preferably elastic threads. The leg elastic members 91 are preferably rubber strips. The number of the elastic threads 91 per extensible side part 9 is preferably about 3 to 12.

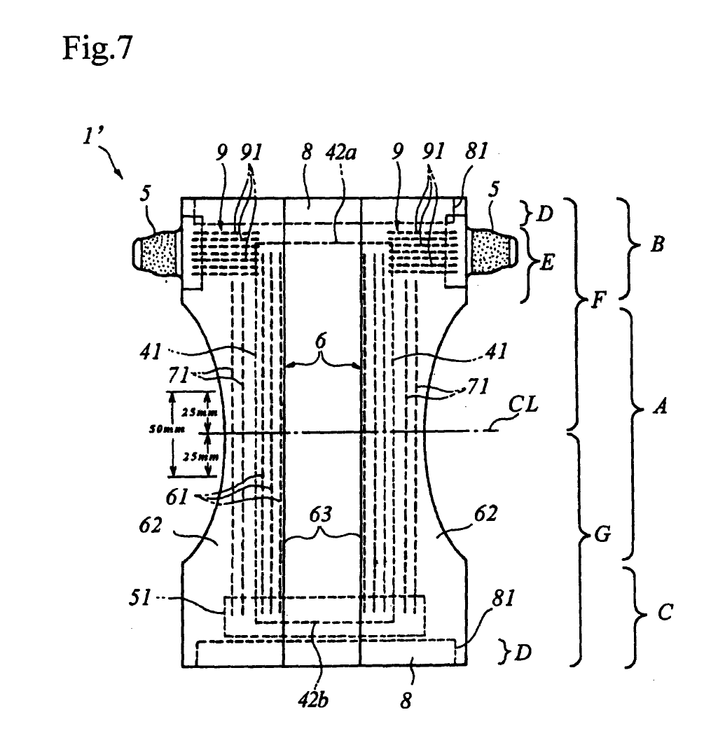

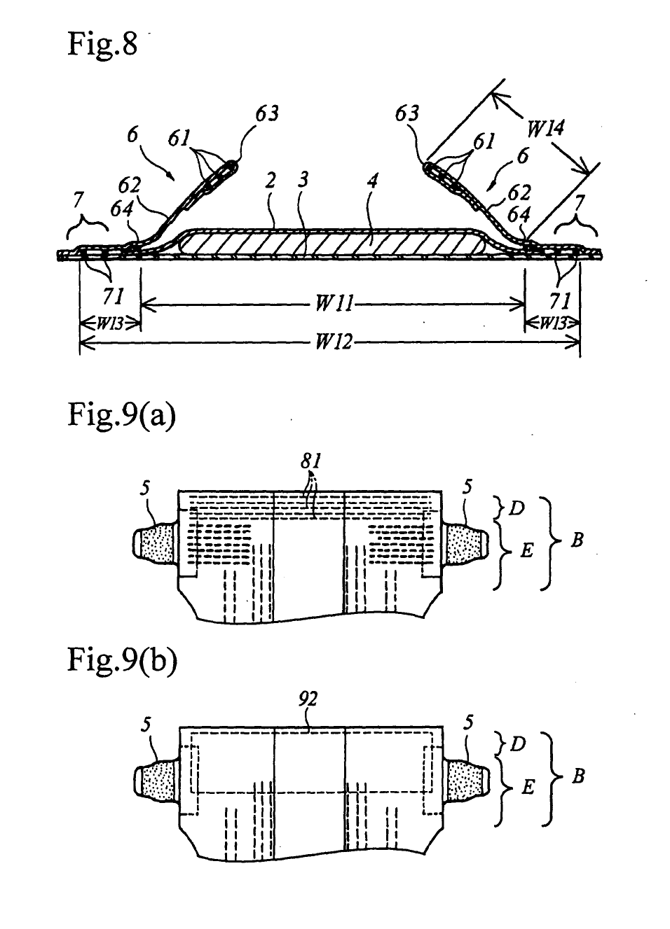

A preferred embodiment of the disposable diaper according to the second aspect of the present invention (hereinafter referred to as a second embodiment of the present invention) will be described with reference to FIGS. 7 and 8. The disposable diaper 1′ shown in these figures is of a fitted type especially suited for babies and infants. The diaper 1′ will be described primarily with respect to aspects different from the above-described diaper 1. Members common to the first and second embodiments are given the same respective reference numbers, and description therefor including their configuration is omitted here.

The disposable diaper 1′ has a pair of standing gathers 6 and a pair of leg gathers 7 provided on each longer side thereof by arranging the respective elastic members.

The pair of leg gathers 7 is formed by disposing a plurality of leg elastic members 71 in an almost linear pattern outward the fixed end 64 of the standing gathers 6. Each of the elastic members 61 and 71 is parallel to the diaper length and extends to the first section B and the second section C.

An elastic member 81 is provided in the waist portion D of each of the first and second sections B and C to form an extensible waist part 8. A plurality of elastic members 91 are provided on each side area of the below-waist portion E in the first section B to form a pair of extensible side parts 9.

In FIG. 7 which is a plan view of the disposable diaper 1′ with all its elastic members in their stretched state, the upper part of the diaper 1′ is designated first section B, and the lower part second section C. The below-waist portion E of the first section B is located below the waist portion D where the waist elastic member 81 is disposed and above the crotch section A (the section applied to the crotch of a wearer and describing an inward arc on both sides thereof in conformity with the wearer's thighs).

The smallest width of the diaper 1′ in the crotch section A is from 100 to 240 mm. Beyond this range, the diaper may be difficult to put through between legs of a wearer in a standing position, or the diaper may leak from the crotch section. For further improved ease of diapering a wearer in a standing position, the smallest width of the crotch section A is preferably from 100 to 230 mm, more preferably from 100 to 210 mm, still even more preferably from 120 to 200 mm.

FIG. 8 is a schematic cross-section of the disposable diaper 1′, taken along a line on which the crotch section is narrowest. In this cross-section, the distance between the fixed ends 64 of the paired standing gathers 6 is taken as a width W11, and the distance between the leg elastic member 71 that is arranged most outward on one side of the diaper 1′ and that on the other side is taken as a width W12. The disposable diaper 1′ has a width ratio W11/W12 of from 0.67 to 0.81, preferably from 0.70 to 0.78. With the smallest width of the diaper 1′ in the crotch section A falling in the above-recited range of from 100 to 240 mm, if the ratio W11/W12 is smaller than 0.67, the space between the standing gathers would be too small to exhibit desired absorbency. If the ratio is greater than 0.81, on the other hand, there may be insufficient pocket left between the standing gathers and the leg gathers, which can cause discharged body waste to flood over and leak.

It is preferred for the disposable diaper 1′ to have the W11/W12 ratio in the range of from 0.67 to 0.81 over an area extending at least 50 mm in the diaper length direction, particularly over an area extending from the smallest width toward opposite sides by at least 25 mm.

In the embodiment shown in FIG. 7, the crotch section A is narrowest at the centerline CL dividing the length of the diaper 1′ into two equal halves. It is desirable that the smallest width of the crotch section A be located at almost the lengthwise middle of the diaper in view of ease of diapering a wearer in a standing position, ease of fastening a diaper on the back side of a crawling baby and for preventing the diaper from sliding down while being worn.

In the disposable diaper 1′ according to the second embodiment, the distance W13 (see FIG. 8) between the fixed end 64 of the standing gathers 6 and the most outward elastic member 71 on each side of the diaper is smaller than the width W14 (see FIG. 8) of the standing gathers 6 at the smallest width of the crotch section A. The width W14 of the standing gathers is the distance from the fixed end 64 to the free end 63, measured on the plane of the sheet 62. If the width W13 is longer than the width W14, the diaper 1′ fails to achieve both the ease of diapering a wearer in a standing position and the absorbing performance. To ensure these properties, a preferred ratio of the width W13 to the width W14 is from 0.45 to 0.83.

It is desirable that the width W13 be smaller than the width W14 over an area extending at least 50 mm in the diaper length direction, particularly over an area extending from the smallest width toward opposite sides by at least 25 mm.

Similarly to the first embodiment, the standing gathers 6 of the disposable diaper 1′ are formed by arranging a low modulus elastic member at a high extension ratio. The elastic member 61 for standing gathers is fixed at a fixing extension ratio (defined above) of 100% or higher, preferably from 100 to 300%, still preferably from 130 to 200%. The tensile load required to extend the standing gathers 6 in its non-fixed state (the state not fixed to the diaper) to an effective extension ratio (defined above) is from 20 to 120 gf, preferably 50 to 100 gf. The tensile load increase rate from 20% extension ratio to the effective extension ratio is 1.0 gf/% or less, preferably 0.7 gf/% or less.

The methods of measuring the extension ratios and the tensile characteristics (tensile load at effective extension ratio and tensile load increase rate) and preferred ranges of these physical properties are the same as described for the disposable diaper 1.

According to the design of the disposable diaper 1′, the substantial width of the crotch section and the distance between the outermost leg gathers are allowed to be reduced. When put on a wearer in a standing position, the diaper 1′ is smoothly inserted through the narrow space under the crotch and pulled up with a good fit of the leg gathers around the thighs. Thus, the diaper 1′ is extremely easy to put on a wearer in a standing position. Moreover, since the standing gathers exhibit high rising capability by virtue of the low modulus elastic members fixed at a high extension ratio, the space between the standing gathers is maintained. As a result, body waste is retained in this space and hardly overruns the standing gathers, and the diaper 1′ exhibits excellent absorbing performance and is substantially leak-proof.

The disposable diaper according to a second aspect of the invention is characterized in that the crotch portion thereof is narrower than a conventional one and yet is leakproof. In designing disposable diapers to be put on a wearer in a lying position, diaper manufacturers may not normally think of reducing the width of the crotch portion near the urine entry points except for the idea that the crotch portion should be curved inward to have a slightly reduced width for giving comfort to a wearer. In contrast, the present inventors have reached an absolutely unique idea that a fitted type disposable diaper having fastening tapes that is designed to be put on a wearer in a standing position would be extremely suited for babies who are in a transitional stage between rolling over and toddling, providing less stress to neither those who have to put the diaper on (for example, parents) as well as to the babies. They have studied this idea regarding how to facilitate diapering a wearer in a standing position without impairing its ability to be leak-proof As a result, they have accomplished the present invention in one aspect by reducing the width of the crotch section in contrast to the generally accepted way of thinking while specifying the configurations of the standing gathers and leg gathers.

Similarly to the disposable diaper 1, two equal sections F and G of the diaper 1′, which are on opposite sides of the centerline CL (see FIG. 7) dividing the length of the diaper 1 in equal halves, have a saturated absorption capacity ratio of from 45/55 to 55/45. By so designing, the diaper 1′ does not substantially leak even when put on in a front-to-back way of diapering as illustrated in FIG. 4. The diaper 1′ is not only suited to a back-to-front way of diapering but also capable of being put on in a front-to-back way with ease and does not substantially leak whichever way of diapering is chosen. In order to obtain theses leak-proof properties irrespective of the way of diapering, the F/G ratio of saturated absorption capacity preferably ranges from 48/52 to 52/48, and the difference in saturated absorption capacity between the F and G sections is preferably as small as possible.

Materials of the members making up the disposable diaper 1′ according to the second embodiment will be described below. The materials for forming the topsheet 2, the backsheet 3, the absorbent member 4, the standing gathers-forming sheet 62, and the landing tape for forming the landing zone 51, and the like are not limited and selected appropriately from known materials commonly used in conventional disposable diapers.

The fastening tape 5 includes a tape having a male member (hook) of a mechanical fastener and a tape having a self-adhesive layer formed by applying a self-adhesive. Where the outer surface of the backsheet 3 is made of an engageable material such as a nonwoven fabric, that surface can serve as a landing zone 51 for the mechanical fasteners.

The standing gathers-forming elastic members 61, the leg elastic members 71, the waist elastic members 81, and the below-waist elastic members 91 are known and conventional. Useful forms of these elastic members include threads (e.g., rubber threads), tapes or strips of some width (e.g., rubber strips), and films. Materials of the elastic members include natural rubber, synthetic rubbers (e.g., styrene-butadiene, butadiene, isoprene or neoprene rubbers), ethylene-vinyl acetate copolymers, extensible polyolefins, and urethane rubber.

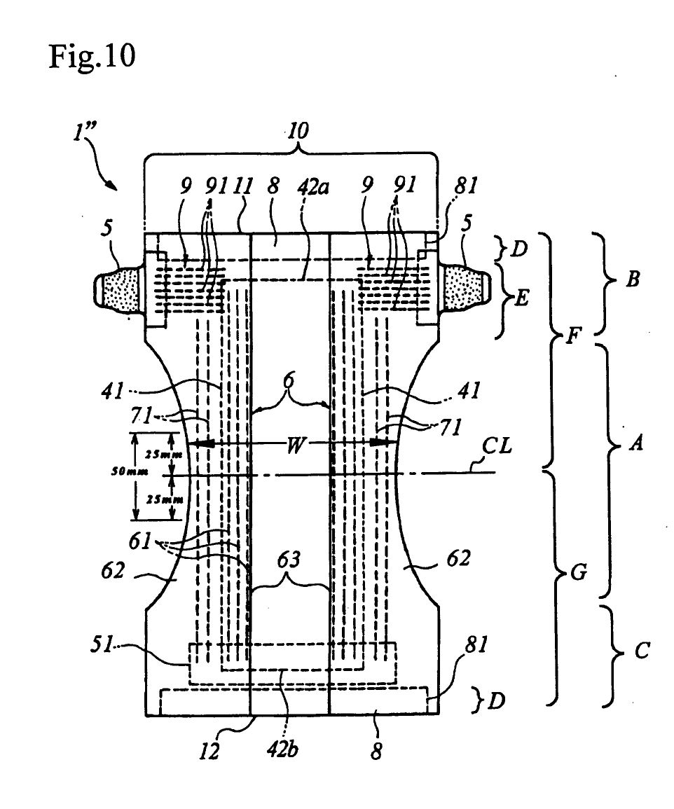

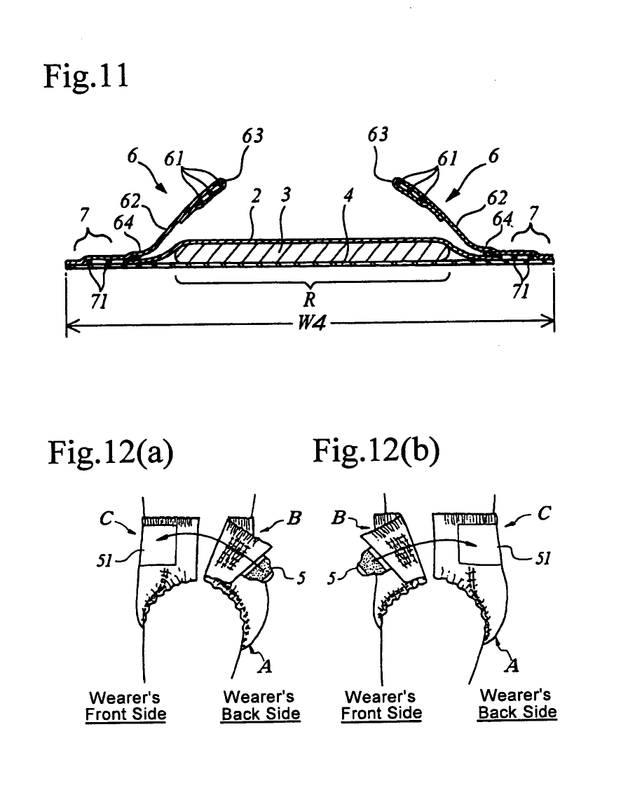

The disposable diaper 1″ shown in FIGS. 10 and 11 is a representation of a preferred embodiment of the third aspect of the present invention (hereinafter referred to as a third embodiment). The diaper 1″ is of a fitted type and designed chiefly for infants.

The diaper 1″ according to a third embodiment will be described primarily with respect to the particulars different from the diaper 1 according to a first embodiment. Members common to the first and third embodiments are given the same respective reference numbers, and description therefor including their configuration is omitted here.

The first section B of the diaper 1″ has a waist portion D and a below-waist portion E. An elastic member 81 is provided in the waist portion D to form an extensible waist part 8. A plurality of elastic members 91 are provided on each side area of the below-waist portion E to form a pair of extensible side parts 9 which are laterally (in the diaper width direction) extensible and contractible. Both extensible side parts 9 are formed between the pair of fastening tapes 5.

The waist portion D constitutes each longitudinal end portion of the diaper 1″ and is held to the waist of a wearer. More specifically, an area from the longitudinal end 11 or 12 to 20 mm inward is called a waist portion D. The diaper 1″ according to this embodiment has the extensible waist part 8 formed by the waist elastic member 81 in the waist portion D in both the first and second sections B and C.

In FIG. 10 that is a plan view of the disposable diaper 1″ with all its elastic members in their stretched flat state, the upper part of the diaper 1″ is designated first section B, and the lower part second section C. The below-waist portion E of the first section B is located below the waist portion D (20 mm wide from the top end downward) and above the crotch section A (the section applied to the crotch of a wearer and describing an inward arc on both sides thereof in conformity with the wearer's thighs). The “side area of the below-waist portion E” as referred to above means the area located within the below-waist portion E and along the longer side of the diaper. The plurality of below-waist elastic members 91 are arranged per extensible side part 9 in parallel to the diaper width direction at prescribed intervals.

The disposable diaper 1″ has a smallest width W4 (see FIG. 11) of from 100 to 240 mm in the crotch section A. A crotch width W is the distance between the side edges of the crotch section A as measured in the diaper width direction with all the elastic members stretched substantially flat as shown in FIG. 10. If the smallest width W4 is smaller than 100 mm, it may be difficult to hold the diaper between the wearer's legs in diapering a wearer in a standing position, and the diaper may fail to assure necessary absorbency and be leak-proof. If it exceeds 240 mm, the ease of diapering a wearer in a standing position and the wearing comfort may be deteriorated. From the standpoint of ease of diapering a wearer in a lying position or a standing position, especially a standing position, the smallest width W4 is preferably from 100 to 230 mm, still preferably from 120 to 200 mm.

It is preferred that the width W of the crotch section A should fall within a range of from 100 to 240 mm over an area extending at least 50 mm in the diaper length direction, particularly over an area extending from the smallest width toward opposite sides by at least 25 mm, as illustrated in FIG. 10. This is preferred for having the diaper held securely between a wearer's legs in a standing position while retaining satisfactory absorbency, being leak-proof and providing comfort. It is still preferred for further improving ease of diapering a wearer in a standing position that the width W be within from 100 to 230 mm over the above-recited area of the crotch section A.

In the disposable diaper 1″ according to a third embodiment, the crotch width W is smallest at the centerline CL (see FIG. 10) dividing the length of the diaper into two equal halves. In this way, the smallest width W4 of the crotch section A is preferably at substantially the lengthwise middle of the diaper.

The disposable diaper 1″ has a lateral flexural stiffness of from 3 to 25 gf/50 mm, preferably from 5 to 20 gf/50 mm, in the region having the absorbent member 4 in the crotch section A (hereinafter referred to as region R, see FIG. 11). If the stiffness of the region R is less than 3 gf/50 mm, it may be difficult to have the diaper held between a wearer's legs while standing during diaper changing. Such a low stiffness, meaning a reduced amount of the absorbent material, can result in a failure to assure necessary absorbency and may be leak-proof. If the stiffness is more than 25 gf/50 mm, the diaper may be difficult to insert between a wearer's legs smoothly while in a standing position and may cause discomfort to the wearer.

The stiffness of the region R should preferably fall within the above-recited range over an area extending at least 50 mm in the diaper length direction, particularly over an area extending from the smallest width toward opposite sides by at least 25 mm.

The stiffness of the region R is measured in the same manner as for the stiffness of the low stiffness region R1 and the high stiffness region R2 of the disposable diaper 1 according to a first embodiment. The specimen for the measurement is cut out of the region R from which standing gathers 6 have been removed with a width of 50 mm parallel to the diaper length direction and a length of 80 mm parallel to the diaper width direction.

Having the above-mentioned configuration, the disposable diaper 1″ of a third embodiment can be easily put on a wearer in a standing position by, for example, the following method of diapering.

As shown in FIGS. 12(a) or 12(b), the diaper 1″ is put through between the wearer's legs so that the crotch section A may come under the wearer's crotch. Because of the above-specified crotch width of the diaper and the above-specified stiffness of the region R, the diaper is then easily pulled up and can be kept in the pulled up state, in which state the fastening tapes are adhered to the landing zone 51.

According to a third embodiment of the invention, because of the specific crotch width of the diaper and the specific stiffness of the region R, the diaper is easy to insert between a wearer's legs while standing, pull up between the legs, and retain such pulled-up state. While the diaper is held in the pulled-up state, one can adhere the fastening tapes to the landing zone 51 with ease. Since the below-waist portion between the fastening tapes are extensible in the diaper width direction, the diaper can be fastened at the right position as in diapering a wearer in a lying position, without having the diaper slide down while fastening.

If the width and the stiffness of the crotch section are reduced without any other change of design, ease of diapering a wearer in a standing position could not be improved without sacrificing the basic performance properties required of a diaper, i.e., sufficient absorbing performance and being leak-proof. In a third aspect of the invention, the inventors have succeeded in improving leak-proof properties and improving ease of diapering, especially ease of diapering a wearer in a standing position, by providing a pair of extensible side parts. Because the pair of the extensible side parts offers an improved fit to a wearer, the absorbent member hardly wrinkles or bunches up thereby allowing the absorbent member to exhibit its absorbency to the maximum. Additionally, the pair of extensible side parts prevents the diaper from sliding down and generating a gap under the wearer's crotch. As a result, the diaper shows sufficient absorbency and is substantially leak-proof even when the width and stiffness of the crotch section is reduced to the above-recited ranges. Thus, the disposable diaper of a third embodiment is easy to put on a wearer in a standing position while retaining high absorbency and reliability against leakage.

The disposable diaper preferably weighs from 20 to 40 g so that it may be held stably in between a wearer's legs while standing and prevented from sliding down even if one looses hold of the diaper during diapering.

According to the above-described method of diapering, diapering can easily be accomplished because one can adhere the fastening tapes to the landing zone while the diaper is stably held in between a wearer's legs while standing.

In a third aspect of the present invention, the tensile load (tensile force) required to extend the below-waist portion E in the first section B to an extension ratio 30% lower than the maximum extension ratio (hereinafter referred to as a tensile load of the below-waist portion E) is from 80 to 500 gf, preferably from 150 to 400 gf. As long as the tensile load of the below-waist portion E falls within this range, the diaper is effectively prevented from sliding down while being put on a wearer in a standing position, and, after being put on, the diaper easily follows the change of contour of the body, e.g., the stomach side of the body and is therefore prevented from sliding down and producing a gap with the crotch of the wearer while being worn. Leakage from the crotch is thus prevented.

The tensile load of the below-waist portion E is measured as follows.

Method of measuring tensile load of below-waist portion E:

The below-waist portion E containing all the below-waist elastic members 91 is cut out across the main body 10 (see FIG. 10). In this particular embodiment, the below-waist portion E is the portion below the 20 mm-wide area from the longitudinal end 11 (in the first section B) inward and between both main body 10's side edges from which the fastening tapes stick out. The cut piece (specimen) is extended in the diaper width direction on a Tensilon tensile tester RTA-100, supplied from Orientec. The specimen is clamped in the jaws set at an initial distance 50 mm shorter than the natural length (initial length) of the specimen as measured with no force applied thereto. The specimen is pulled at a crosshead travel speed of 300 mm/min up to its maximum extension length. The term “maximum extension length” as used herein means the length of the specimen from which all the elastic members have been removed, that is, the length of the specimen with no gathers. The maximum extension ratio P (%) of the specimen is obtained from [(maximum extension length−initial length)/initial length]×100. The tensile load (tensile force) at an extension ratio 30% lower than the maximum extension ratio P is read.

The below-waist elastic members which can be used in a third embodiment include elastic threads having a thickness of 650 dtex or smaller and elastic strips. Considering that the below-waist portion E fits the body to prevent the whole diaper from sliding down without being too tight around the body at the extensible side parts, it is preferred to use elastic threads having a thickness of from 200 to 600 dtex. Although fine elastic members give a reduced tightening feel, they are difficult to fix to a sheeting material. Hence, it is preferred that the elastic threads are previously fixed in between two films to prepare an extensible sheet, which is adhered to the main body 10 of the diaper. By so doing, the elastic threads should never be separated and drawn even when the extensible side parts are suddenly pulled by an unintended force during diaper changing.

The standing gathers 6 of the disposable diaper 1″ are formed by arranging a low modulus elastic member at a high extension ratio similarly to those of the first and second embodiments. The fixing extension ratio (defined above) of the standing gathers is 100% or higher, preferably from 100 to 300%, still preferably from 130 to 200%.

The tensile characteristics of the standing gathers 6 (either one of the pair) are such that the tensile load required to extend the standing gathers 6 in its non-fixed state (the state not fixed to the diaper, that is, the state after being cut off the diaper) to an effective extension ratio (defined above) is from 20 to 120 gf, preferably from 50 to 100 gf and that a tensile load increase rate from 20% extension ratio to the effective extension ratio is 1.0 gf/% or lower, preferably 0.7 gf/% or lower. The methods of measuring the extension ratios and the tensile characteristics (tensile load at effective extension ratio and tensile load increase rate), preferred ranges of these physical properties, and means for achieving the recited ranges of these properties are the same as described for the disposable diaper 1.

The disposable diaper 1″ of a third embodiment has low stiffness in the part of the region having the absorbent member 4 and has a pair of standing gathers 6 formed by fixing low modulus elastic members at a high extension ratio. Therefore, it is extremely easy to put on a wearer whether the wearer is in a lying position or a standing position. The standing gathers exhibit satisfactory rising capabilities because of the low modulus and the high fixing extension ratio and therefore successfully maintain a sufficient space enough to prevent leaks. Improvements on leakage and ease of diapering have thus been achieved.

The diaper 1″ has improved ease of diapering a wearer in a standing position while retaining safety against leaks. It is smoothly placed through a narrow space between the legs of a wearer who is standing and held to the same position of the wearer's body as intended. Since it is no more necessary to extremely increase the lateral extension length or the width of the first section B in an attempt to improve the ease of diapering a wearer in a standing position, there is no impairment of ease of diapering a wearer in a lying position.

Similarly to a first embodiment, the disposable diaper 1″ according to the third embodiment has an F/G saturated absorption capacity ratio of from 45/55 to 55/45. As a result, the diaper 1″ hardly leaks whether it is put on in a back-to-front way or a front-to-back way. In order to secure safety against leakage irrespective of the way of diapering, the F/G ratio of saturated absorption capacity preferably ranges from 48/52 to 52/48, and the difference in saturated absorption capacity between the F and G sections is preferably as small as possible. FIG. 12(a) shows the disposable diaper 1″ being put on an infant in a standing position with the first section B on the back of the wearer (in a back-to-front way of diapering). FIG. 12(b) shows the diaper 1″ being put on an infant in a standing position with the first section B on the stomach (in a front-to-back way of diapering).

Materials of the members making up the disposable diaper 1″ are the same as those described with respect to the diapers 1 and 1′ described supra.

While the present invention has been described with particular reference to its preferred embodiments, it should be understood that the invention is not construed as being limited thereto, and various modifications can be made therein without departing from the spirit and scope thereof.

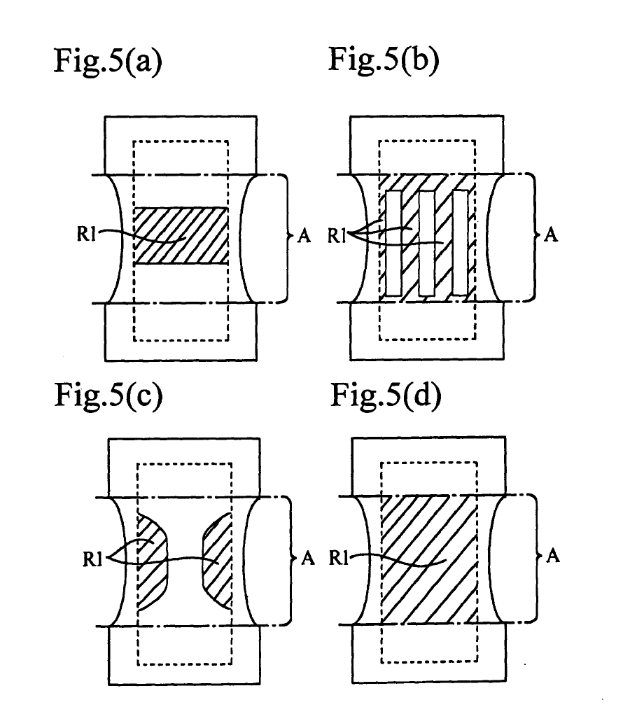

For example, the following modifications can be made to a first embodiment. While the diaper 1 has the low stiffness region R1 discretely provided on both sides of the region R in the diaper length direction, the low stiffness region R1 can be arranged according to the configurations shown in FIGS. 5(a) to 5(d). FIG. 5(a) shows a configuration in which a low stiffness region R1 is continuously provided in the middle portion of the region R over the whole width of the absorbent member 4. FIG. 5(b) depicts a configuration in which a low stiffness region R1 is provided all over the region R except for discretely arranged high stiffness regions. FIG. 5(c) shows another stiffness configuration. FIG. 5(d) displays a configuration in which all the region R is a low stiffness region R1.

While the diaper 1 has an extensible part on each side area of the below-waist portion E of the first section B (a pair of side extensible parts 9), a laterally extensible part may be additionally provided between the pair of side extensible parts 9 in the below-waist portion E either as an extension from the side extensible parts 9 or discretely from the side extensible parts 9.

The leg elastic members 71 may be arranged to depict a curve along each arched side edge of the crotch section A. The sections, portions, parts, and the other members constituting the disposable diaper of the present invention are also subject to variation in shape or configuration.

The following modifications can be made to the diaper 1′ of the second embodiment. The elastic strip 81 provided in the waist portion D to make the waist extensible part 8 may be replaced with a plurality of elastic threads 81, which are provided over substantially the whole width of the waist portion D as shown in FIG. 9(a). In this modification, the elastic threads may previously be fixed on a soft film or a like support to prepare a composite elastic sheet, which is then fixed to the waist portion D. It is also possible that the waist elastic member 81 and the below-waist elastic members 91 may be replaced with a single elastic strip 92 having a width of 20 to 50 mm, which is provided on an area from the waist end inward and between the pair of fastening tapes 5 as shown in FIG. 9(b).

The above-mentioned modification that replaces the elastic strip 81 with a plurality of elastic threads 81 is also applicable to the diaper 1″ of a third embodiment. In this case, too, the elastic threads 81 may previously be fixed on a soft film or a like support to prepare a composite elastic sheet, which is then fixed to the waist portion D.

The disposable diaper according to the present invention is, while applicable to a wide range of wearers from newborns to adults, is particular suited for babies and infants, especially those who are prone to hate being diapered and those who have the ability to pull themselves up into a standing position.

In the description given above, some particulars of an embodiment may have been omitted for the sake of avoiding redundancy. This omission can be complemented by the corresponding description of other embodiments. Particular characteristics of an embodiment may be applied to other embodiments appropriately. Particulars of every embodiment are appropriately interchangeable between embodiments.

The present invention will now be illustrated in greater detail with reference to Examples, but it should be understood that the invention is not construed as being limited thereto.

[I] Examples of First Aspect of the Invention:

### EXAMPLE 1

A disposable diaper shown in FIGS. 1 to 3 was prepared. In Table 1 are shown the smallest width W4 in the crotch section A, the width W2 of the region R, the width W3 and flexural stiffness of the high stiffness region R2, the width W1 and bending stiffness of each of the low stiffness regions R1, the fixing extension ratio of the standing gathers, the tensile characteristics (i.e., effective extension ratio (=fixing extension ratio-30%), tensile load at the effective extension ratio, and tensile load increase rate) of the standing gathers in their non-fixed state, and the section F to section G ratio of saturated absorption capacity. The standing gathers on each side were formed of two elastic strips adhered to a nonwoven fabric sheet.

The absorbent member was fabricated by the aforementioned method (1). The absorbent member had a stiff part in the widthwise middle and a soft part on each longer side thereof. The disposable diaper prepared had otherwise the same members in the same configuration as in a standard product hereinafter described.

### EXAMPLE 2

A disposable diaper was prepared in the same manner as in Example 1, except for changing the width W3 and bending stiffness of the high stiffness region R2 and the width W1 and bending stiffness of the low stiffness region R1 as shown in Table 1.

### EXAMPLES 3 AND 4

Disposable diapers were prepared in the same manner as in Example 1, except for using an absorbent member having a low bending stiffness all over the entire area, the width and the bending stiffness of which are shown in Table 1.

All the diapers of Examples 1 to 3 had the following properties. The lateral tensile load of the below-waist portion E in the first section B at a given extension ratio was greater than that of the waist portion D in the first section B at the same extension ratio. Each of the side extensible parts measured about 40 mm in the diaper width direction in its contracted state. When the two fastening tapes were pulled apart in the diaper width direction to extend the extensible side parts, the tensile load at a middle extension length ranged from 30 to 150 gf

Standard Product:

A commercially available fitted type disposable diaper (Merries Morenai-Fit Nobichizimi Gather, Size M for infants, available from Kao Corp.) was used as a standard product for comparison. The dimensional and physical characteristics of this product are shown in Table 1. When the fastening tapes were pulled apart in the diaper width direction, the tensile load at a middle extension length ranged from 30 to 150 gf.

### COMPARATIVE EXAMPLE 1

A disposable diaper having the same structure as the standard product was prepared, except for using an absorbent member having a stiff part in the widthwise middle and a soft part in each longer side thereof

### COMPARATIVE EXAMPLE 2

A disposable diaper having the same structure as the standard product was prepared, except for using an absorbent member having a high stiffness all over the entire area thereof

### COMPARATIVE EXAMPLE 3

A disposable diaper was prepared in the same manner as in Example 3, except that no elastic member was disposed in the below-waist portion.

FIG. 6 is a graph showing the relationship between the extension ratio and tensile stress (load) of standing gathers in their non-fixed state. Curve 1 represents the diapers of Examples 1 to 3, and curve 2 represents the standard product.

TABLE 1

| | | | | | | | | Non-fixed State Tensile Characteristics | | | |
|---|---|---|---|---|---|---|---|---|---|---|---|
| | | | Region R2 | | Region R1 | | | | Tensile Load at | | |
| | | | | Bending | | Bending | Fixing | Effective | Effective | Tensile Load | F/G Saturated |
| | W4 | W2 | W3 | Stiffness | W1 | Stiffness | Extension | Extension | Extension Ratio | Increase Rate | Absorption |
| | (mm) | (mm) | (mm) | (gf/cm) | (mm) | (gf/cm) | Ratio (%) | Ratio (%) | (gf) | (gf/%) | Capacity Ratio |
| Standard | 244 | 100 | 100 | 28.4 | — | | 60 | 30 | 40 | 1.2 | 45/55 |
| Product | | | | | | | | | | | |
| Example 1 | 220 | 100 | 70 | 44.5 | 15 | 21.2 | 110 | 80 | 48 | 0.5 | 50/50 |
| Example 2 | 220 | 100 | 60 | 44.5 | 20 | 21.2 | 110 | 80 | 48 | 0.5 | 50/50 |
| Example 3 | 220 | 100 | — | | 100 | 6.2 | 110 | 80 | 48 | 0.5 | 50/50 |
| Example 4 | 220 | 100 | — | | 100 | 24.0 | 110 | 80 | 48 | 0.5 | 50/50 |
| Comparative | 220 | 100 | 40 | 67.3 | 30 | 10.5 | 60 | 30 | 40 | 1.2 | 50/50 |
| Example 1 | | | | | | | | | | | |

| | | | | | | | | | | |
|---|---|---|---|---|---|---|---|---|---|---|
| Comparative | 220 | 100 | 100 | 67.3 | — | 60 | 30 | 40 | 1.2 | 50/50 |
| Example 2 | | | | | | | | | | |

| | | | | | | | | | | |
|---|---|---|---|---|---|---|---|---|---|---|
| Comparative | 220 | 100 | — | 100 | 24.0 | 110 | 80 | 48 | 0.5 | 50/50 |
| Example 3 | | | | | | | | | | |

The disposable diapers of Examples 1 to 3 and Comparative Examples 1 and 2 were evaluated for ease of diapering and resistance to sliding down in motion in accordance with the following test methods.

1) Ease of Diapering (in Back-to-Front Way)

Eleven people who used or have used disposable diapers during child care were asked to put the diapers of the Examples, Comparative Examples and the standard product on their baby in a lying position and a standing position in a back-to-front way (the first section B of the diaper was held to the baby's back, and the fastening tapes were adhered to the landing zone on the stomach side) and to score the ease of diapering on a 100-point scale. The panelists scored the standard product, followed by the other diapers. The difference obtained by subtracting the score of the standard product from that of a tested diaper was taken as the score of that diaper indicative of ease of diapering in comparison with the standard product. The total of the scores given to each diaper by the eleven panelists are shown in Table 2.

2) Motion of Sliding Down on the Body

A simulated infant hip model was used for testing. The model is shaped to mimic the hips and buttocks of an infant and is designed to perform a walking movement in a standing position. A diaper was put on the model in a standing position in a back-to-front way so that the top waist end of the diaper was positioned 70 mm below the top end of the model, at which the waist of the model measured 42 cm and in which the distance between the tips of the fastening tapes adhered to the landing zone was 13 cm.

The diapered model was operated to make a 5-minute walking movement at a pace of 140 steps per minute. After stopping the walking movement, the sliding distance (the distance between the original position of the top end of the diaper and the position after the walking movement) was measured. The results obtained are shown in Table 2.

The diaper of Comparative Example 2 was not tested because it did not fit the crotch of the model.

TABLE 2

| | | | Sliding |
|---|---|---|---|
| | Ease of Diapering | | Distance |
| | on Lying Model | on Standing Model | (mm/5 min) |
| Standard Product | 0 | 0 | 20 |
| Example 1 | 30 | 95 | 13 |
| Example 2 | 70 | 115 | 15 |
| Example 3 | 35 | 45 | 16 |
| Comp. Example 1 | −105 | −35 | 19 |
| Comp. Example 2 | −420 | −270 | not measured |

The standard product and the diapers of Example 4 and Comparative Example 3 were tested for sliding distance in the same manner as described above, except that the diapers were put on backward (in a front-to-back way). The results obtained are shown in Table 3 below.

TABLE 3

| | Sliding Distance |
|---|---|
| | (mm/5 min) |
| Standard Product | 23 |
| Example 4 | 19 |
| Comp. Example 3 | 27 |

It is understood that the disposable diaper according to a first aspect of the invention is easy to put on a wearer whether the wearer is in a standing position or a lying position. It is also seen that they are reliable against leaks while worn when put on a wearer in a standing position.

[II] Examples of a Second Aspect of the Invention

### EXAMPLES 5 AND 6 AND COMPARATIVE EXAMPLES 4 AND 5

Disposable diapers shown in FIGS. 7 and 8 were prepared. Table 4 shows the smallest width of the crotch section, the distance W11, out of the smallest width, between the fixed ends 64 of the paired standing gathers 6, the distance W12, out of the smallest width, between the leg elastic member 71 that is the most outward on one longer side of the diaper and that on the other longer side, the W11/W12 ratio, the distance W13, out of the smallest width, between the fixed end 64 of the standing gathers 6 and the most outward elastic member 71 on each side of the diaper, the width W14 of the standing gathers (on each side) at the smallest width, the fixing extension ratio of the standing gathers, and the tensile characteristics of the standing gathers in their non-fixed state.

In the diapers of Examples 5 and 6, the W11/W12 ratio ranged from 0.67 to 0.81 over a diaper length of 50 mm or longer, and the distance W13 was smaller than the width W14 over a diaper length of 50 mm or longer.

The relationship between the extension ratio and tensile stress (load) in the standing gathers in their non-fixed state are shown in FIG. 6, in which curve 1 represents the diapers of Examples 5 and 6, and curve 2 the diaper of Comparative Example 4.

TABLE 4

| | | | | | | | | Non-fixed State Tensile Characteristics | | |
|---|---|---|---|---|---|---|---|---|---|---|
| | | | | | | | | | Tensile Load at | |
| | | | | | | Smallest | Fixing | Effective | Effective | Tensile Load |
| | W11 | W12 | W11/ | W13 | W14 | Crotch | Extension | Extension Ratio | Extension Ratio | Increase rate |
| | (mm) | (mm) | W12 | (mm) | (mm) | Width (mm) | Ratio (%) | (%) | (gf) | (gf/%) |
| Example 5 | 145 | 205 | 0.71 | 30 | 36.5 | 220 | 110 | 80 | 48 | 0.5 |
| Example 6 | 140 | 180 | 0.78 | 20 | 36.5 | 200 | 110 | 80 | 48 | 0.5 |
| Comparative | 150 | 228 | 0.66 | 39 | 43.5 | 254 | 60 | 30 | 40 | 1.2 |
| Example 4 | | | | | | | | | | |
| Comparative | 100 | 120 | 0.83 | 10 | 36.5 | 120 | 110 | 80 | 48 | 0.5 |
| Example 5 | | | | | | | | | | |

The diapers of Examples 5 and 6 and Comparative Examples 4 and 5 were evaluated for ease of diapering a wearer in a standing position, leak-proof, properties and space retention for retaining soft tools in accordance with the following test methods. The results obtained are shown in Table 5.

1) Ease of Diapering a Wearer in a Standing Position

The diaper was put in a back-to-front manner (the fastening tapes were adhered on the landing zone on the wearer's front side) on an infant model in a standing position. A diaper that was capable of being put on with its front top end positioned on the level of the navel was judged “good”. A diaper that slid down while being fastened so that the front top end was positioned below the navel was judged “bad”.

2) Leak-Proof Properties While in Motion

A simulated infant hip model was used for testing. The model is shaped to mimic the hips and buttocks of an infant and is designed to perform a walking movement in its standing position and to discharge artificial urine through a tube from the crotch. A disposable diaper was put on the infant hip model in a back-to-front way with its front waist end level to the navel. The model was operated to make a 5-minute walking movement at a pace of 150 steps per minute. After stopping the walking movement, 80 g of artificial urine was poured through the tube at a rate of 5 g/sec, and the model was again operated to make the same walking movement for an additional 5 minutes. Then the model was laid on its side, and 40 g of artificial urine was poured at a rate of 5 g/sec. At the point when the pouring was completed, the occurrence of any leakage was inspected with the naked eye. Where no leaks were observed, an additional 40 g portion of the artificial urine was poured. Pouring another 40 g portion of artificial urine was repeated until a leak occurred. The total amount of the artificial urine poured until leakage occurred was taken as a measure of leak-proof properties while in motion.

3) Space Retention for Retaining Soft Stools

An infant model was used, which was capable of making a walking movement in a standing position, opening and closing the legs in a sitting position, changing between a sitting position and a standing position, and discharging artificial urine and artificial stools through tubes from the crotch.

A diaper was put on the model in a back-to-front way with its front waist end on the level of the model's navel. The model was operated to make a 5-minute walking action at a pace of 150 steps/min. After stopping the walking movement, 80 g of artificial urine was poured through one of the tubes at a rate of 5 g/sec, and the model was again operated to make the same walking movement for an additional 5 minutes. Then the model was made to sit up with its legs open, and 160 g of artificial stool was poured at a rate of 10 g/sec. The model was operated to repeat cycles of walking, changing into a sitting position, and opening and closing the legs for a total of 5 minutes. The disposable diaper was removed to see the spread of the artificial stool.

The diaper which had allowed the artificial stool to overrun the standing gathers was judged “bad”, and the one which had not was judged “good”.

TABLE 5

| | Ease of Diapering | Leak-proof | Space |
|---|---|---|---|
| | on Wearer in a | Properties While | Retention for |
| | standing position | in Motion (g) | Soft Stools |
| Example 5 | Good | 160 | good |
| Example 6 | Good | 160 | good |
| Comparative | Bad | 120 | bad |
| Example 4 | | | |
| Comparative | Good | 120 | bad |
| Example 5 | | | |

The disposable diaper according to a second aspect of the present invention is shown to be easy to place on a wearer in a standing position and excellent in absorbing performance and is substantially leak-proof.

[III] Examples of Third Aspect of the Invention

### EXAMPLE 7

Disposable diapers shown in FIG. 10 were prepared. Each of the extensible side parts 9 measured 9 cm in the diaper width direction. The smallest width W4 of the crotch section A was 220 mm. The region R (the region having the absorbent member in the crotch section) had a bending stiffness of 20 gf/50 mm. The extensible side parts 9 were formed using elastic threads having a thickness of 560 dtex. The tensile load of the below-waist portion E was 300 gf.

The width W of the crotch section A was in a range of from 100 to 240 mm over an area extending 50 mm or longer in the diaper length direction. The diaper weighed 25 g. The F/G saturated absorption capacity ratio was 49/51.

### COMPARATIVE EXAMPLE 6

A disposable diaper was prepared in the same manner as in Example 7, except that the extensible side parts were not provided, the smallest width W4 was 255 mm, and the absorbent member used had a stiffness of 40 gf/50 mm.

### COMPARATIVE EXAMPLE 7

A disposable diaper was prepared in the same manner as in Example 7, except that the region R had a bending stiffness of 40 gf/50 mm.

FIG. 6 is a graph showing the relationship between the extension ratio and tensile stress (load) in standing gathers in their non-fixed state (the state removed from the diaper). Curve 1 represents the diapers of Example 7 and Comparative Examples 6 and 7. The standing gathers of Example 7 were fixed at a fixing extension ratio of 110%, had a tensile load of 48 gf at the effective extension ratio (110-30=80%), and showed a tensile load increase rate of 0.5 gf/% from 20% extension ratio to the effective extension ratio.

The diapers of Example 7 and Comparative Example 6 were tested for ease of diapering a standing infant by a panel of 10 members as follows. The diaper was put on an infant model in a standing position with the fastening tapes adhered on the stomach side (back-to-front way of diapering). As a result, the diaper of Example 7 received affirmative support of all the members with regard to ease of diapering a wearer in a standing position.

The diapers of Example 7 and Comparative Examples 6 and 7 were evaluated for leakage and resistance to sliding down while being worn as follows. The results obtained are shown in Table 6. A simulated infant hip model was used for testing. The model was shaped to mimic the hips and buttocks of an infant and is designed to perform a walking movement in a standing position and to discharge artificial urine from its crotch through a tube. A diaper was put in a back-to-front way on the model in a standing position with its front top end level with the model's navel. The diapered model was operated to make a 5-minute walking movement at a pace of 150 steps per minute. After stopping the walking movement, 80 g of artificial urine was poured through the tube at a rate of 5 g/sec, and the model was again operated to make the same walking movement for an additional 5 minutes. The sliding distance (the distance between the model's navel to the top end of the diaper) was measured.

Then the model was laid on its side, and 40 g of artificial urine was poured at a rate of 5 g/sec. At the time point when the pouring was completed, the occurrence of any leak occurred was inspected with the naked eye. Where no leaks was observed, an additional 40 g portion of the artificial urine was poured. Pouring an additional 40 g portion of artificial urine was repeated until a leak occurred. The total amount of the artificial urine poured until leakage was taken as a measure of leak-proof properties while in motion.

TABLE 6

| | | Comparative | Comparative |
|---|---|---|---|
| | Example 7 | Example 6 | Example 7 |
| Leak-proof Properties | 160 | 120 | 120 |
| While in Motion (g) | | | |
| Sliding Distance (mm) | 20 | 40 | 20 |

The fitted type disposable diaper according to a third aspect of the invention is easy to put on a wearer in a standing position and yet superior in absorbency and is leak-proof. The present invention also provides in a third aspect a novel method of diapering with a fitted type disposable diaper, which enables a one to easily change diapers on a wearer in a standing position.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

This application claims the priority of Japanese Patent Application Nos. 2002-218960 filed Jul. 26, 2002, 2002-218963 filed Jul. 26, 2002, 2002-218965 filed Jul. 26, 2002, 2003-27632 filed Feb. 4, 2003 and 2003-27633 filed Feb. 4, 2003, which are incorporated herein by reference.

What is claimed is:

1. A disposable diaper comprising a liquid permeable topsheet, a liquid impermeable backsheet, a liquid retentive absorbent member interposed between the topsheet and the backsheet, a pair of longitudinal side portions and a pair of longitudinal end sections,
a fastening tape provided on each side edge of one of the longitudinal end sections thereof and a laterally extensible side part provided on each side area of a below-waist portion of the longitudinal end section having the fastening tapes, and standing gathers provided in each of the longitudinal side portions thereof, wherein
a region R having the absorbent member in a crotch section disposed therein having a low stiffness region R1 having a flexural stiffness of 25 gf/50 mm or less in the width direction of the diaper,
the standing gathers being formed by fixing an elastic member having a fixing extension ratio of 100% or higher, and
the standing gathers on each longitudinal side portion having tensile characteristics, which as measured in a state of the gathers not being fixed to the diaper, wherein the tensile load required to extend the standing gathers to an effective extension ratio which is 30% lower than a fixing extension ratio, is from 20 to 120 gf, and the rate of increase of the tensile load required to extend from an extension ratio of 20% up to the effective extension ratio is 1.0 gf/% or lower.

2. The disposable diaper according to claim 1, wherein the region R comprises a high stiffness region R2 having a width of at least 50 mm across the diaper and a flexural stiffness of 25 gf/50 mm or higher in the width direction of the diaper.

3. The disposable diaper according to claim 1, wherein the low stiffness region R1 exists along each longer side edge of the diaper in the region R.

4. The disposable diaper according to claim 1, wherein the extensible side parts each have a natural length of 20 mm or more as measured in the width direction of the diaper in a state being removed from the diaper and contracted, and the tensile characteristics of the extensible side parts are such that, when the fastening tapes are pulled apart in the width direction of the diaper to extend the extensible side parts, the tensile load at a middle extension length is from 30 to 300 gf.

5. The disposable diaper according to claim 1, wherein the width of the crotch section as its smallest width is from 100 to 240 mm.

6. The disposable diaper according to claim 1, wherein two portions on opposite sides of a centerline dividing the length of the diaper in equal halves have a saturated absorption capacity ratio of from 45/55 to 55/45.

7. A disposable diaper for babies and infants comprising a liquid permeable topsheet, a liquid impermeable backsheet, a liquid retentive absorbent member interposed between the topsheet and the backsheet, and a fastening tape provided on each side edge of one of a pair of longitudinal end sections thereof and having a pair of standing gathers and a pair of leg gathers each formed by fixing respective elastic members in each of a pair of longitudinal side portions thereof, wherein
a smallest width of the crotch section is 100 to 240 mm,
a ratio of a distance W11 between a pair of fixed ends of the pair of standing gathers measured at the smallest width of a crotch section to a distance W12 between each elastic member that is arranged most outward in each of the respective leg gathers, and with both distances being measured at the smallest width of the crotch section, W11/W12, is from 0.67 to 0.81,
a distance W13 between each of the fixed ends of the pair of standing gathers and each of the elastic members that are arranged most outward in the pair of leg gathers as measured at the smallest width of the crotch section is smaller than a width W14 of the standing gathers on each side as measured at the smallest width of the crotch section,
the standing gathers being formed by fixing an elastic member having a fixing extension ratio of 100% or higher, and
the standing gathers on each longitudinal side portion having tensile characteristics, which as measured in a state of the gathers not being fixed to the diaper, wherein the tensile load required to extend the standing gathers to an effective extension ratio, which is 30% lower than a fixing extension ratio is from 20 to 120 gf, and the rate of increase of the tensile load required to extend from an extension ratio of 20/% to the effective extension ratio is 1.0 gf/% or lower.

8. The disposable diaper according to claim 7, wherein the ratio W11/W12 is in the range of from 0.67 to 0.81 over an area extending at least 50 mm in a length direction of the diaper.

9. The disposable diaper according to claim 7, wherein the distance W13 is smaller than the width W14 over an area extending at least 50 mm in a length direction of the diaper.

10. A disposable diaper comprising a liquid permeable topsheet, a liquid impermeable backsheet, a liquid retentive absorbent member interposed between the topsheet and the backsheet, and a fastening tape provided on each side edge of one of a pair of the longitudinal end sections thereof, and a landing zone where the fastening tape can be fixed on an outer side of the other longitudinal end section of the pair, and a laterally extensible side part provided on each side area of a below-waist portion of the longitudinal end section having the fastening tapes, wherein
a tensile load required to extend the below-waist portion of the longitudinal end section having the fastening tapes in the diaper width direction to an extension ratio that is 30% lower than a maximum extension ratio is from 80 to 500 gf,
a smallest width of a crotch section of the diaper is from 100 to 240 mm, and
a region having the absorbent member in the crotch section has a flexural stiffness of from 3 to 25 gf/50 mm in a width direction of the diaper.

11. The disposable diaper according to claim 10, wherein the crotch section possesses a width that is in a range of from 100 to 240 mm in an area extending at least 50 mm in a length direction of the diaper.

12. The disposable diaper according to claim 10, which weighs from 20 to 40 g.

13. The disposable diaper according to claim 10, wherein two portions on opposite sides of a centerline dividing the length of the diaper in equal halves have a saturated absorption capacity ratio of from 45/55 to 55/45.

14. A method of diapering a wearer in a standing position with a disposable diaper comprising a liquid permeable topsheet, a liquid impermeable backsheet and a liquid retentive absorbent member interposed between the topsheet and the backsheet and having a fastening tape provided on each side edge of one of a pair of longitudinal end sections thereof, a landing zone where the fastening tape can be fixed on the outer side of the other longitudinal end section, and a laterally extensible side part provided on each side area of the below-waist portion of the longitudinal end section having the fastening tapes, wherein
a smallest width of the crotch section is from 100 to 240 mm, and a region having the absorbent member in the crotch section has a bending stiffness of from 3 to 25 gf/50 mm in a width direction of the diaper,
the method comprising the steps of putting the disposable diaper between a standing wearer's legs, pulling up the disposable diaper to the wearer's crotch so that the diaper is retained to the crotch and fixing the fastening tapes onto the landing zone while the diaper is retained to the crotch.
